(19) United States
(12) Patent Application Publication
(10) Pub. No.: US 2022/0079570 A1
(43) Pub. Date: Mar. 17, 2022

(54) ENDOSCOPE TREATMENT TOOL

(71) Applicant: OLYMPUS CORPORATION, Tokyo (JP)

(72) Inventor: Junji SHIONO, Tokyo (JP)

(73) Assignee: OLYMPUS CORPORATION, Tokyo (JP)

(21) Appl. No.: 17/534,705

(22) Filed: Nov. 24, 2021

Related U.S. Application Data
(63) Continuation of application No. PCT/JP2019/026925, filed on Jul. 8, 2019.

(51) Int. Cl. A61B 10/06; A61B 17/29; A61B 10/04

(52) U.S. Cl. CPC A61B 10/06; A61B 17/29; A61B 2017/2901; A61B 2017/2939; A61B 10/04

(57) ABSTRACT

Provided is an endoscope treatment tool including: a sheath; a distal-end support member that is secured to a distal end of the sheath and that has a rotation shaft extending in an axial direction orthogonal to a longitudinal axis of the sheath; a pair of gripping members that are linked to the rotation shaft in a pivotable manner; and an operation wire that is linked to the gripping members, wherein the distal-end support member includes: a pair of frame pieces that extend in a direction of the longitudinal axis with a spacing therebetween in a direction orthogonal to the longitudinal axis and that include through-holes through which the rotation shaft passes; and a plate that includes a bearing to which the rotation shaft is fitted and an abutting portion protruding radially outward in the sheath, and wherein the plate extends into a space between the rotation shaft and the sheath.

### CROSS-REFERENCE TO RELATED APPLICATIONS

This is a continuation of International Application PCT/JP2019/026925 which is hereby incorporated by reference herein in its entirety.

### TECHNICAL FIELD

The present invention relates to an endoscope treatment tool.

### BACKGROUND ART

There is a known endoscope treatment tool in which a pair of forceps cups are supported by a distal-end support member secured to a distal end of a flexible sheath so as to be freely opened/closed centered on a support shaft, the forceps cups being distal-end treatment members of biopsy forceps (for example, see Patent Literature 1).

The distal-end support member includes slots facing each other with a gap therebetween. A linking mechanism for opening/closing the forceps cups is disposed in the slots. The linking mechanism is driven as a result of operating an operation wire, which is inserted into the flexible sheath, in an advancing/retracting manner, and opens/closes the forceps cups in a beak-like manner.

### CITATION LIST

### Patent Literature

{PTL 1} Japanese Unexamined Patent Application, Publication No. 2002-58675

### SUMMARY OF INVENTION

An aspect of the present invention is an endoscope treatment tool including: a sheath; a distal-end support member that is secured to a distal end of the sheath and that has a rotation shaft extending in an axial direction orthogonal to a longitudinal axis of the sheath; a pair of gripping members that are linked to the rotation shaft in a pivotable manner; and an operation wire that is linked to the gripping members, wherein the distal-end support member includes: a pair of frame pieces that extend in a direction of the longitudinal axis with a spacing therebetween in a direction orthogonal to the longitudinal axis and that include through-holes through which the rotation shaft passes; and a plate that includes a bearing to which the rotation shaft is fitted and an abutting portion protruding radially outward in the sheath, and wherein the plate extends into a space between the rotation shaft and the sheath.

### BRIEF DESCRIPTION OF DRAWINGS

FIG. 1 is an overall configuration diagram showing a state in which a pair of gripping members of an endoscope treatment tool according to an embodiment of the present invention are closed.

FIG. 2 is a longitudinal sectional view partially showing a distal-end portion of the endoscope treatment tool in FIG. 1.

FIG. 3 is a side view partially showing the distal-end portion of the endoscope treatment tool in FIG. 1.

FIG. 4 is an overall configuration diagram showing a state in which the pair of gripping members of the endoscope treatment tool in FIG. 1 are open.

FIG. 5 is a longitudinal sectional view partially showing the distal-end portion of the endoscope treatment tool in FIG. 4.

FIG. 6 is a longitudinal sectional view showing a state in which a buckling prevention member of the endoscope treatment tool in FIG. 1 and an end surface of a coil tube are abutted against each other.

FIG. 7 is a partial longitudinal sectional view showing a modification of the endoscope treatment tool in FIG. 1.

FIG. 8 is a side view partially showing another modification of the endoscope treatment tool in FIG. 1.

FIG. 9 is a partial lateral sectional view showing another modification of the endoscope treatment tool in FIG. 1.

FIG. 10 is a partial lateral sectional view showing another modification of the endoscope treatment tool in FIG. 1.

FIG. 11 is a partial perspective view showing the endoscope treatment tool in FIG. 10.

### DESCRIPTION OF EMBODIMENT

An endoscope treatment tool 1 according to an embodiment of the present invention will be described below with reference to the drawings.

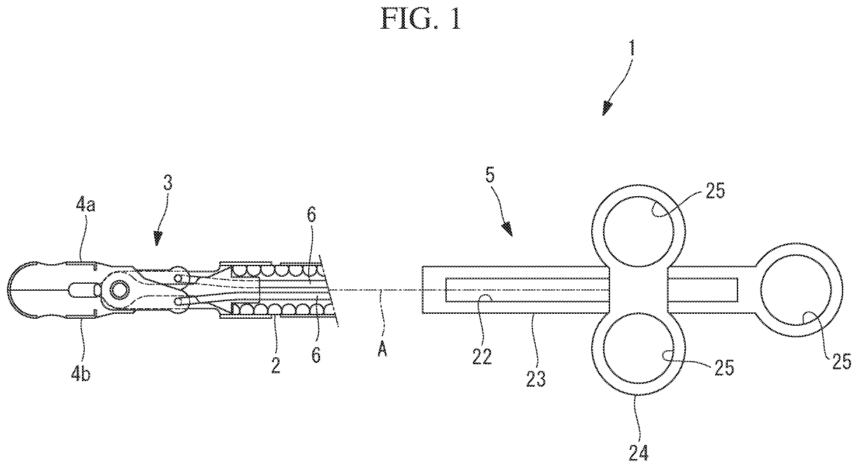

As shown in FIG. 1, the endoscope treatment tool 1 according to this embodiment includes: a coil tube (sheath) 2; a distal-end support member 3 that is secured to a distal end of the coil tube 2; a pair of gripping members 4a and 4b supported by the distal-end support member 3; an operating portion 5 provided at a proximal end of the coil tube 2; and an operation wire 6 that links the operating portion 5 with the gripping members 4a and 4b.

In the figure, reference sign 7 is a resin cover tube that covers an external surface of the coil tube 2. The coil tube 2 is formed by tightly winding, in a spiral manner, a steel wire made of stainless steel or the like. The coil tube 2 and the cover tube 7 have certain external-diameter dimensions that allow insertion thereof into an endoscope channel (not shown).

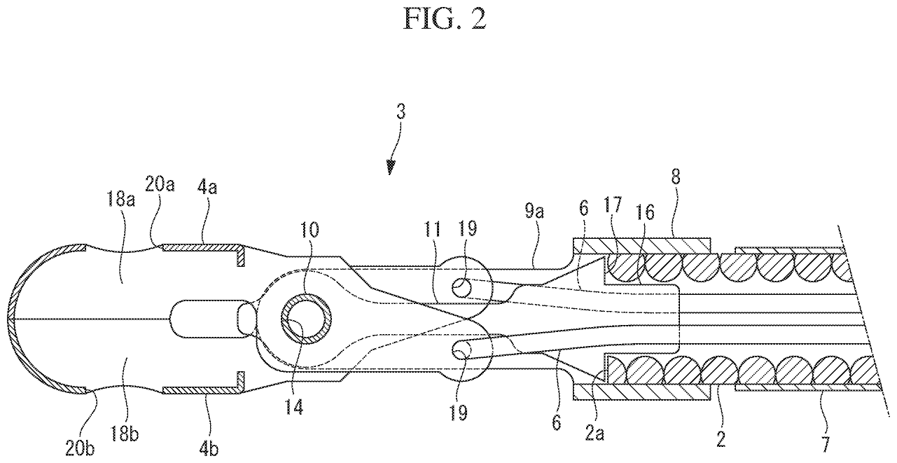

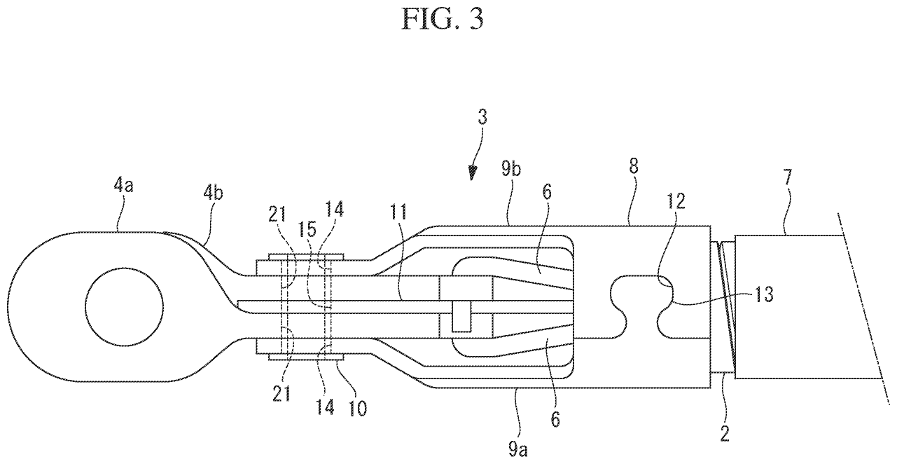

As shown in FIGS. 2 and 3, the distal-end support member 3 includes: a cylindrical stationary portion 8; a pair of frame pieces 9a and 9b that extend from the distal end of the stationary portion 8 and that are disposed at positions at which the frame pieces face each other in a radial direction with a spacing therebetween; a rotation shaft 10 that extends in a direction orthogonal to an axial direction in which the frame pieces 9a and 9b extend and that links distal-end portions of the frame pieces 9a and 9b with each other; and a buckling prevention member 11.

The stationary portion 8 and the pair of frame pieces 9a and 9b of the distal-end support member 3 are integrally formed by punching a metal thin plate by means of stamping and by bending the punched product. The stationary portion 8 is cylindrically formed by bending a strip-shaped portion and by engaging, in a circumferential direction, a depressed portion 12 and a protruding portion 13 that are at the two ends of the strip-shaped portion and that have complementary shapes.

The stationary portion 8 has an inner diameter that is nearly equivalent to the external diameter of the coil tube 2, is disposed at a position at which an outer circumference of an end surface 2a of the coil tube 2 is surrounded over the entire circumference thereof as a result of the coil tube 2 being fitted to the stationary portion 8, and is secured to the coil tube 2 by means of welding or the like.

The respective frame pieces 9a and 9b extend farther toward a distal side than the distal end of the coil tube 2.

In addition, the lateral sectional shapes of the respective frame pieces 9a and 9b are formed in an arc shapes in portions thereof to be connected to the stationary portion 8. Through-holes 14 through which the rotation shaft 10 passes are individually formed in distal-end portions of the respective frame pieces 9a and 9b.

As shown in FIG. 2, the buckling prevention member 11 is formed from a strip-shaped metal thin plate (plate) including, in one end portion thereof, a through-hole 15 through which the rotation shaft 10 passes in the plate-thickness direction. The one end portion of the buckling prevention member 11 forms a bearing in which the rotation shaft 10 fits into the through-hole 15. The width of the other end portion of the buckling prevention member 11, in a direction orthogonal to the longitudinal axes of the respective frame pieces 9a and 9b and the center axis of the rotation shaft 10, has a slightly smaller width dimension than the inner diameter of the coil tube 2. In addition, the other end portion of the buckling prevention member 11 is provided with: an insertion end portion 16 that is inserted into the coil tube 2; and an abutting portion (abutting surface) 17 that is formed so as to have a greater width than that of the insertion end portion 16 and that is disposed at a position at which the abutting portion abuts against the end surface 2a of the coil tube 2. In other words, the abutting portion 17 protrudes radially outward in the coil tube 2.

In a state in which a tensile force is not applied to the operation wire 6, described later, the abutting portion 17 is disposed with a minute gap in the direction parallel to a longitudinal axis A of the coil tube 2 with respect to the end surface 2a of the coil tube 2, as shown in FIG. 2. In addition, the abutting portion 17 has a shape in which the width thereof decreases, toward a distal end of the buckling prevention member 11, from positions at which the abutting portion protrudes outward in the width direction from both sides of the insertion end portion 16 in the width direction thereof. The maximum width of the abutting portion 17 is configured so as to be slightly smaller than the inner diameter of the stationary portion 8.

As shown in FIG. 3, the gripping member 4a, 4b is formed, for example, as a result of stamping a metal thin plate, in a shape having: a through-hole 21 through which the rotation shaft 10 passes at the center thereof; a cup-shaped gripping portion 18a, 18b at one end thereof; and, at the other end, an attaching hole 19 to which the operation wire 6 is attached. In the figure, reference signs 20a and 20b are holes that are provided in the gripping portions 18a and 18b and that allow the tissue to partially escape.

In order to assemble the distal-end support member 3 and the pair of gripping members 4a and 4b, the through-holes 21 of the pair of gripping members 4a and 4b, the through-hole 15 of the buckling prevention member 11, and the through-holes 14 of the pair of frame pieces 9a and 9b are aligned with each other, and the pair of gripping members 4a and 4b, between which the buckling prevention member 11 is sandwiched, are inserted between the pair of frame pieces 9a and 9b. In this state, the rotation shaft 10 in the form having a flange at one end thereof is made to pass through the through-holes 14, 15, and 21 of the respective members 4a, 4b, 9a, 9b, and 11 from the frame piece 9a on one side, and the other end of the rotation shaft that has passed through the frame piece 9b on the other side is crimped. Accordingly, as shown in FIG. 3, the pair of gripping members 4a and 4b are attached to the distal-end support member 3 so that the gripping members can be opened/closed in a beak-like manner. In addition, the rotation shaft 10 may be made to pass through the through-holes from the frame piece 9b on the other side and may be crimped after being made to pass through the frame piece 9a on the one side.

The operating portion 5 includes: a rod-shaped operating-portion body 23 that is attached to a proximal end of the coil tube 2 and in which a groove 22 is formed in a longitudinal direction thereof; and a slider 24 that is supported by the operating-portion body 23 so as to be slidable along the groove 22. The operating-portion body 23 and the slider 24 are provided with finger-hooking portions 25 in which an operator hooks his/her fingers.

In the example shown in FIGS. 1 to 3, the endoscope treatment tool 1 includes two operation wires 6. Proximal ends of the operation wires 6 are respectively connected to the slider 24, distal ends of the operation wires 6 are respectively connected to the attaching holes 19 of the pair of gripping members 4a and 4b.

The operation of the thus-configured endoscope treatment tool 1 according to this embodiment will be described below.

In order to collect tissue in a body by employing the endoscope treatment tool 1 according to this embodiment, the endoscope treatment tool 1 is inserted into the body via a channel of an endoscope inserted into the digestive tract.

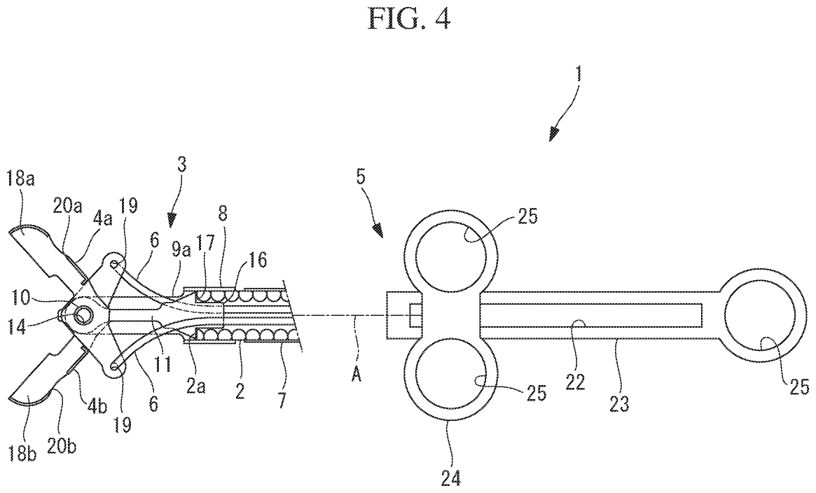

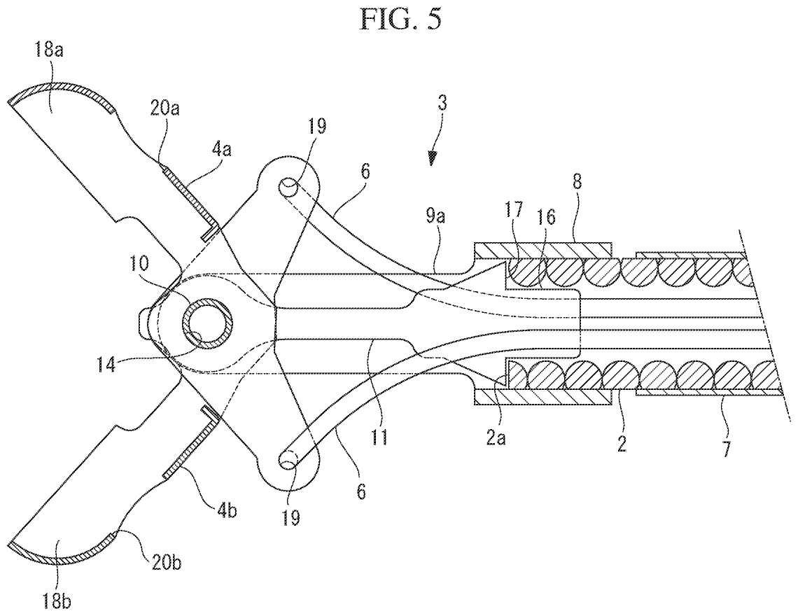

An operator aligns the pair of gripping members 4a and 4b of the endoscope treatment tool 1 with the position of the tissue to be collected while checking an image acquired by means of the endoscope. In this state, the slider 24 is advanced toward the distal end of the operating-portion body 23 along the groove 22 with respect to the operating-portion body 23 of the operating portion 5. Accordingly, as shown in FIGS. 4 and 5, the pair of gripping members 4a and 4b are pressed by the operation wires 6, the pair of gripping members 4a and 4b are pivoted about the rotation shaft 10, and the pair of gripping portions 18a and 18b are placed in the open state.

Then, the tissue to be collected is disposed between the open pair of gripping portions 18a and 18b, and the slider 24 is retracted toward the proximal end of the operating-portion body 23 along the groove 22 with respect to the operating-portion body 23 of the operating portion 5. Accordingly, as shown in FIG. 1, the pair of gripping members 4a and 4b are pivoted about the rotation shaft 10, and the pair of gripping portions 18a and 18b are closed.

Because the tissue to be collected is sandwiched between the pair of gripping portions 18a and 18b in this state, when the operator applies a force to the operating portion 5 in the direction in which the slider 24 is further retracted with respect to the operating-portion body 23, it is possible to cut out and collect the tissue by causing the distal ends of the gripping portions 18a and 18b to cut into the tissue. In the case in which the amount of the tissue accommodated in the gripping portions 18a and 18b is excessive, the excess tissue is allowed to protrude from holes 20a and 20b provided in the gripping portions 18a and 18b.

In this case, when the operator applies a high force to the operating portion 5 in order to cut out the tissue, the pair of gripping members 4a and 4b are pulled toward the proximal end due to the high tensile forces applied to the operation wires 6; therefore, high compression forces also act on the pair of frame pieces 9a and 9b via the rotation shaft 10. Thus, the pair of frame pieces 9a and 9b subjected to the compression forces are minutely elastically deformed so as to be compressed in the direction parallel to the longitudinal axis A of the coil tube 2.

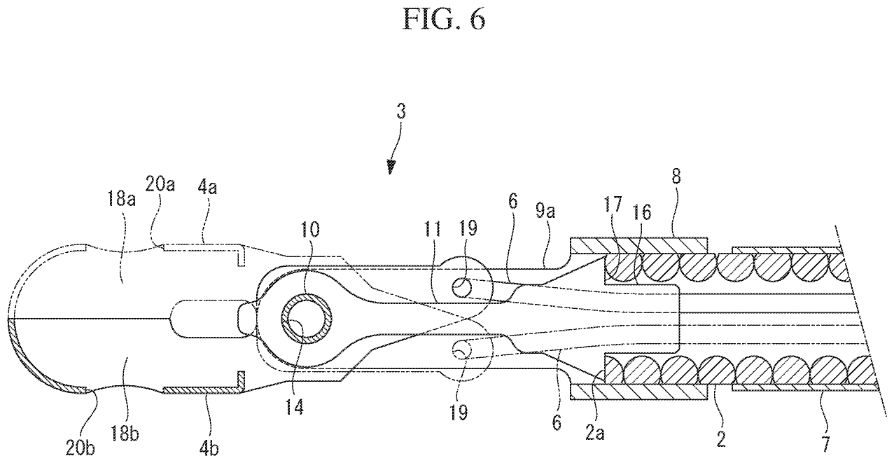

Accordingly, because the position of the rotation shaft 10 is minutely moved toward the proximal end in the direction parallel to the longitudinal axis A of the coil tube 2, the abutting portion 17 of the buckling prevention member 11, which has been disposed with a gap with respect to the end surface of the coil tube 2, is made to abut against the end surface 2a of the coil tube 2, as shown in FIG. 6. In other words, at this point, the rotation shaft 10 is simultaneously supported by the pair of frame pieces 9a and 9b and the buckling prevention member 11; therefore, the compression forces are dispersed and excessive forces are prevented from acting on the frame pieces 9a and 9b.

As has been described above, with the endoscope treatment tool 1 according to this embodiment, simply by disposing the buckling prevention member 11 formed from a metal thin plate between the rotation shaft 10 and the end surface 2a of the coil tube 2, it is possible to reduce the compression forces acting on the pair of frame pieces 9a and 9b and to prevent buckling thereof. Therefore, there is an advantage in that, even in the case in which the pair of frame pieces 9a and 9b are formed by stamping a metal thin plate, it is possible to support the gripping members 4a and 4b with high strength.

In other words, there is an advantage in that it is possible to provide the endoscope treatment tool 1 in which the strength thereof is enhanced without forming thick frame pieces 9a and 9b by cutting a block-shaped metal piece, even with a simple, low-cost configuration in which a metal thin plate is simply stamped.

In addition, with this embodiment, in the state in which high tensile forces are not applied to the operation wires 6, and the frame pieces 9a and 9b are not elastically deformed, a minute gap is formed between the abutting portion 17 and the end surface 2a of the coil tube 2. Accordingly, it is possible to set the precision of machining the buckling prevention member 11 and the precision of assembling the coil tube 2 and the distal-end support member 3 to be low, and thus, there is an advantage in that it is possible to reduce manufacturing costs. In addition, it is possible to save time in joining the buckling prevention member 11 with the end surface 2a of the coil tube 2.

In addition, as a result of the position at which the abutting portion 17 abuts against the end surface 2a of the coil tube 2 being surrounded by the cylindrical stationary portion 8 of the distal-end support member 3, the position is prevented from being exposed to the exterior. Accordingly, it is possible to prevent tissue from being sandwiched in the gap between the abutting portion 17 and the end surface 2a of the coil tube 2.

In addition, when fitting the distal end of the coil tube 2 into the stationary portion 8 of the distal-end support member 3, it suffices to fit these components until reaching a position at which the end surface 2a of the coil tube 2 lightly abuts against the abutting portion 17; therefore, it is possible to utilize the abutting portion 17 as a guide during assembly.

In addition, the buckling prevention member 11 is formed so that the width thereof decreases from the abutting portion 17 toward the distal end of the buckling prevention member 11; therefore, the buckling prevention member does not protrude radially outward farther beyond the external diameter of the stationary portion 8 and it is possible to prevent interference with peripheral tissue or the like.

In addition, the insertion end portion 16 provided in the buckling prevention member 11 is inserted into the coil tube 2; therefore, it is possible to position the abutting portion 17 at the position at which the abutting portion reliably abuts against the end surface 2a of the coil tube 2.

Note that, in this embodiment, the abutting portion 17 of the buckling prevention member 11 is abutted against the end surface 2a of the coil tube 2; alternatively, however, the abutting portion 17 may be abutted against the distal end of the stationary portion 8. In addition, a gap is provided between the abutting portion 17 and the end surface 2a of the coil tube 2, and the abutting of the components is achieved by elastic deformation of the frame pieces 9a and 9b; however, a configuration without the gap may be employed. In addition, the abutting portion 17 may be secured to the end surface 2a of the coil tube 2 or the stationary portion 8 by means of welding or the like.

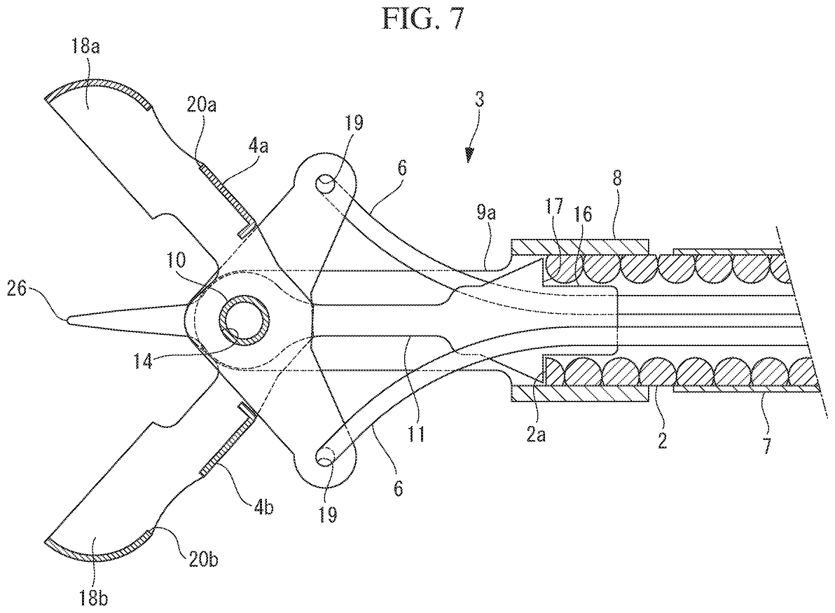

In addition, as shown in FIG. 7, the distal end of the buckling prevention member 11 may include a needle portion 26 that protrudes into a space between the pair of gripping members 4a and 4b. By employing such a configuration, when sandwiching tissue by means of the gripping portions 18a and 18b, it is possible to pierce the tissue with the needle portion 26 and to prevent the gripping portions 18a and 18b from being shifted from a portion that needs to be collected. As a result of integrally providing the needle portion 26 in the buckling prevention member 11 by using a thin metal plate, it is possible to reduce the number of components.

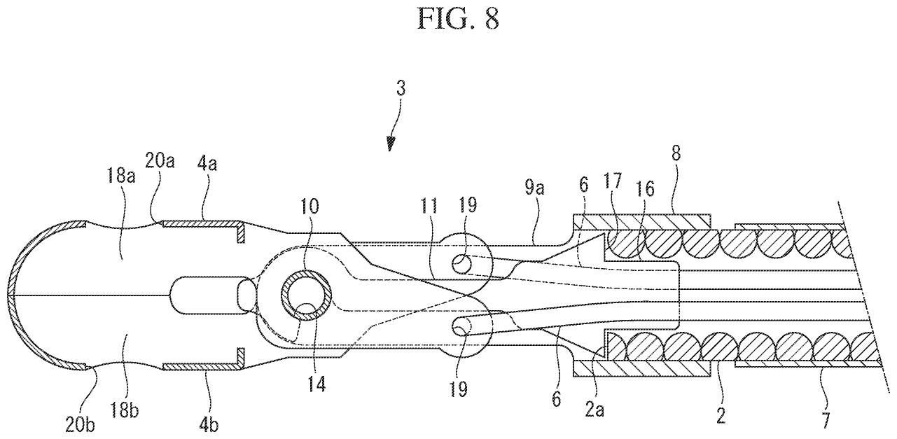

In addition, in this embodiment, the buckling prevention member 11 including the through-hole 15 at the one end portion thereof has been described as an example of the bearing; alternatively, however, as shown in FIG. 8, a hook-shaped portion formed by cutting out a portion of the one end portion of the buckling prevention member 11 so that the through-hole 15 and the exterior become continuous may be employed.

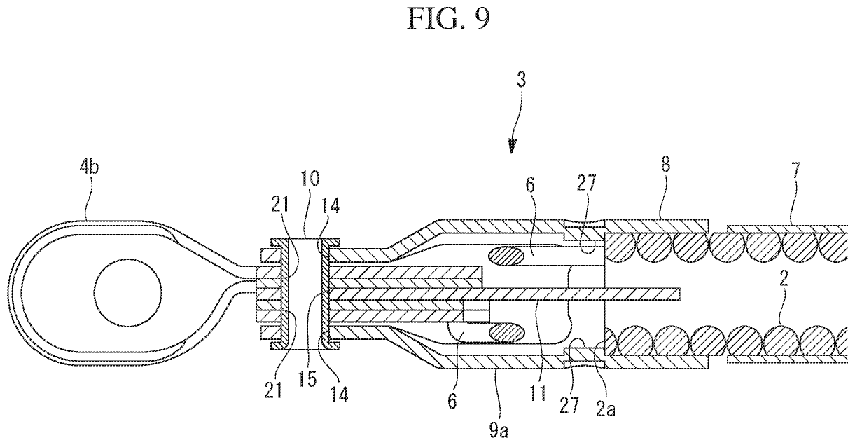

In addition, as shown in FIG. 9, in this embodiment, the stationary portion 8 may include a protrusion 27 that is formed by making a portion of an inner circumferential surface thereof protrude in a direction intersecting the longitudinal axis of the stationary portion 8. Accordingly, when fitting the distal end of the coil tube 2 into the stationary portion 8 of the distal-end support member 3, it suffices to fit the coil tube until the coil tube reaches a position at which the end surface 2a of the coil tube 2 abuts against the protrusion 27 of the stationary portion 8, and it is possible to facilitate the positioning.

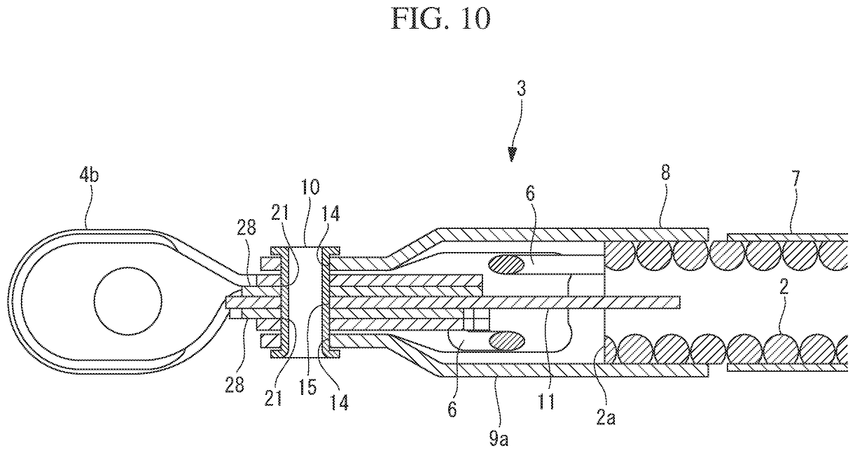

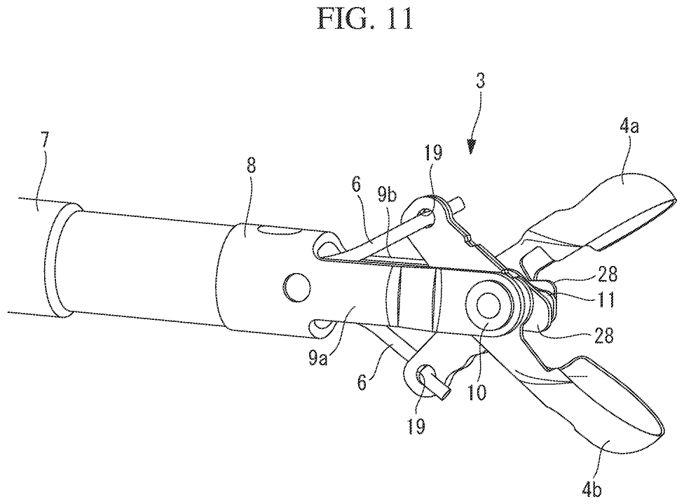

In addition, as shown in FIGS. 10 and 11, in this embodiment, protrusions 28 that extend from the rotation shaft 10 toward the distal ends of the gripping members 4a and 4b may be provided in proximal end portions of the gripping members 4a and 4b. In this case, because the buckling prevention member 11 has a certain area extending toward the distal ends of the gripping members 4a and 4b, the contact area between the gripping members 4a and 4b having the protrusions 28 and the buckling prevention member 11 increases. Accordingly, it is possible to suppress shifting of the gripping members 4a and 4b with respect to each other.

The following aspects can be also derived from this embodiment.

An aspect of the present invention is an endoscope treatment tool including: a sheath; a rotation shaft that extends in an axial direction orthogonal to a longitudinal axis of the sheath; a pair of gripping members that are linked to the rotation shaft in a pivotable manner; an operation wire that is linked to the gripping members; and a plate that includes a bearing to which the rotation shaft is fitted and an abutting portion protruding radially outward in the sheath, wherein the plate extends into a space between the rotation shaft and the sheath.

In this aspect, when the operation wire is pulled in the state in which the pair of gripping members are closed, due to the tensile force transmitted by the operation wire, the rotation shaft supporting the gripping members is pulled toward the proximal end of the sheath and the plate disposed between the rotation shaft and the sheath receives a compression force in a direction of the longitudinal axis.

Therefore, because the compression force acting on the rotation shaft is received by the plate, the compressive stresses applied to the individual members are reduced, and it is possible to provide an endoscope treatment tool in which the strength thereof is enhanced with a simple configuration.

In addition, as a result of fitting the rotation shaft to the bearing of the plate, it is possible to attach the plate between the rotation shaft and the sheath in a simple manner.

The above-described aspect may include: a distal-end support member that is secured to a distal end of the sheath and that supports the gripping members so as to be pivotable about the axis; and an operating portion that is provided at a proximal end of the sheath, wherein the operation wire may be inserted into the sheath in the direction of the longitudinal axis, may link the gripping members with the operating portion, may be advanced/retracted in the direction of the longitudinal axis as a result of the operating portion being operated, and may transmit a tensile force that causes the gripping members to be pivoted, wherein the distal-end support member may include a pair of frame pieces that extend in the direction of the longitudinal axis with a spacing therebetween in a direction orthogonal to the longitudinal axis, wherein the rotation shaft may extend in the axial direction and may link distal-end portions of the frame pieces with each other, and wherein the plate may receive a compression force in the direction of the longitudinal axis when the tensile force acts on the operation wire.

With this configuration, an operator operates the operating portion while checking an image acquired by means of an endoscope. Accordingly, the operation wire is pulled toward the proximal end in the direction of the longitudinal axis, the tensile force is transmitted by the operation wire to the pair of gripping members disposed at the distal end of the sheath, and the gripping members are pivoted about the axis of the rotation shaft. As a result of closing the gripping members in the state in which tissue is disposed between the pair of gripping members, the tissue is gripped.

When the operator further pulls the operation wire by means of the operating portion in the state in which the pair of gripping members are closed, due to the tensile force transmitted by the operation wire, the rotation shaft of the distal-end support member supporting the gripping members is pulled toward the proximal end of the sheath; therefore, the frame pieces receive compression forces in the direction of the longitudinal axis. At this time, with this aspect, the plate disposed between the rotation shaft and the sheath receives a compression force in the direction of the longitudinal axis.

Therefore, because the compression force acting on the rotation shaft is received by the pair of frame pieces and the plate in a dispersed manner, the compressive stresses applied to the individual members are reduced, and it is possible to prevent buckling of the frame pieces. In other words, as a result of enhancing the strength with a simple configuration without increasing the thicknesses of the frame pieces and without using a high-strength material, it is possible to prevent the endoscope treatment tool from breaking during the operation of the gripping members.

In addition, in the above-described aspect, the abutting portion may abut against an end surface of the sheath when a tensile force acts on the operation wire.

With this configuration, the abutting portion abuts against the end surface of the sheath when the tensile force acts on the operation wire, and the abutting portion receives a compression force as a result of being sandwiched between the rotation shaft and the end surface of the sheath.

In addition, in the above-described aspect, the distal-end support member may include a stationary portion that secures the pair of frame pieces to the sheath, and the stationary portion and the pair of frame pieces may be integrally formed by bending a metal thin plate.

With this configuration, it is possible to integrally form the stationary portion and the pair of frame pieces by bending a metal thin plate, and it is possible to manufacture the distal-end support member at low cost. As a result of securing the distal-end support member to the sheath by means of the stationary portion, it is possible to provide the pair of frame pieces so as to extend from the end surface of the sheath along the longitudinal axis.

In addition, in the above-described aspect, the abutting portion may abut against the stationary portion when the tensile force acts on the operation wire.

With this configuration, the abutting portion abuts against the stationary portion when the tensile force acts on the operation wire, and the abutting portion receives a compression force as a result of being sandwiched between the rotation shaft and the end surface of the sheath with the stationary portion interposed therebetween.

In addition, in the above-described aspect, the plate may include an insertion end portion that extends in the direction of the longitudinal axis and that is inserted into the sheath.

With this configuration, as a result of inserting the insertion end portion of the plate into the sheath, it is possible to position the abutting portion in the radial direction so as not to be shifted from the sheath or the stationary portion.

In addition, in the above-described aspect, the stationary portion may be cylindrically formed so as to surround an area radially outside an end surface of the sheath, and the abutting portion may abut against the end surface of the sheath radially inside the stationary portion when the tensile force acts on the operation wire.

With this configuration, as a result of surrounding, by means of the cylindrical stationary portion, the portion in which the abutting portion abuts against the end surface of the sheath, it is possible to prevent the portion from being exposed to the exterior. Accordingly, it is possible to prevent tissue from being sandwiched between the abutting portion and the end surface of the sheath.

In addition, in the above-described aspect, the plate may include a needle portion that protrudes into a space between the pair of gripping members.

With this configuration, as a result of piercing and securing, by means of the needle portion, the tissue to be sandwiched between the pair of gripping members, it is possible to accurately grip a site of the tissue that needs to be collected. Also, as a result of integrating such a needle portion with the plate, it is possible to reduce the number of components.

### REFERENCE SIGNS LIST

1 endoscope treatment tool
2 coil tube (sheath)
2a end surface
3 distal-end support member
4a, 4b gripping member
5 operating portion
6 operation wire
8 stationary portion
9a, 9b frame piece
10 rotation shaft
11 buckling prevention member (plate)
16 insertion end portion
17 abutting portion
26 needle portion
A longitudinal axis

1. An endoscope treatment tool comprising:
a sheath;
a distal-end support member that is secured to a distal end of the sheath and that has a rotation shaft extending in an axial direction orthogonal to a longitudinal axis of the sheath;
a pair of gripping members that are linked to the rotation shaft in a pivotable manner; and
an operation wire that is linked to the gripping members,
wherein the distal-end support member comprises:
a pair of frame pieces that extend in a direction of the longitudinal axis with a spacing therebetween in a direction orthogonal to the longitudinal axis and that include through-holes through which the rotation shaft passes; and
a plate that includes a bearing to which the rotation shaft is fitted and an abutting portion protruding radially outward in the sheath, and
wherein the plate extends into a space between the rotation shaft and the sheath.

2. The endoscope treatment tool according to claim 1, further comprising:
an operating portion that is provided at a proximal end of the sheath,
wherein the operation wire is inserted into the sheath in the direction of the longitudinal axis, links the gripping members with the operating portion, is advanced/retracted in the direction of the longitudinal axis as a result of the operating portion being operated, and transmits a tensile force that causes the gripping members to be pivoted,
wherein the rotation shaft links distal ends of the frame pieces with each other, and
wherein the plate receives a compression force in the direction of the longitudinal axis when the tensile force acts on the operation wire.

3. The endoscope treatment tool according to claim 1, wherein the abutting portion abuts against an end surface of the sheath when a tensile force acts on the operation wire.

4. The endoscope treatment tool according to claim 2, wherein the distal-end support member includes a stationary portion that secures the pair of frame pieces to the sheath, and
the stationary portion and the pair of frame pieces are integrally formed by bending a metal thin plate.

5. The endoscope treatment tool according to claim 4, wherein the abutting portion abuts against the stationary portion when the tensile force acts on the operation wire.

6. The endoscope treatment tool according to claim 3, wherein the plate includes an insertion end portion that extends in the direction of the longitudinal axis and that is inserted into the sheath.

7. The endoscope treatment tool according to claim 4, wherein the stationary portion is cylindrically formed so as to surround an area radially outside an end surface of the sheath, and
the abutting portion abuts against the end surface of the sheath radially inside the stationary portion when the tensile force acts on the operation wire.

8. The endoscope treatment tool according to claim 1, wherein the plate includes a needle portion that protrudes into a space between the pair of gripping members.
